(19) United States
(12) Patent Application Publication
(10) Pub. No.: US 2020/0309796 A1
(43) Pub. Date: Oct. 1, 2020

(54) ASSAY, METHOD AND TREATMENT OF ALPHA-SYNUCLEINOPATHIES

(71) Applicant: H. Lundbeck A/S, Valby (DK)

(72) Inventors: Ibrahim John Malik, Valby (DK); Pekka Kallunki, Valby (DK); Karina Fog, Valby (DK)

(21) Appl. No.: 16/955,984

(22) PCT Filed: Dec. 19, 2018
(86) PCT No.: PCT/EP2018/085898

§ 371 Date: Jun. 19, 2020

(30) Foreign Application Priority Data
Dec. 21, 2017 (DK) PA201700738

(51) Int. Cl. G01N 33/68; G01N 33/542; G01N 33/58; C07K 16/18

(52) U.S. Cl. CPC G01N 33/6896; G01N 33/542; G01N 2800/2835; C07K 16/18; G01N 33/582

(57) ABSTRACT

A method of detecting alpha-synuclein in a sample of a subject comprising the steps of a) Obtaining a sample from a subject,5 b) Applying the sample on a luminescence assay, and c) Optionally, comparing the results with a control

### FIELD OF THE INVENTION

The present invention relates to an assay that enables diagnosis and efficient treatment of alpha-synucleinopathies such as Parkinson's Disease patients by assessing and monitoring the alpha-synuclein levels in patients.

### BACKGROUND OF THE INVENTION

Parkinson's disease (PD), Parkinson's disease with dementia (PDD), dementia with Lewy bodies (DLB) and multiple systems atrophy (MSA) are examples of neurodegenerative disorders with a-synuclein brain pathology. PD is the most common movement disorder and is characterized by tremor, bradykinesia or difficulty to initiate movements, rigidity and impairment of balance also referred to as postural instability. PD is believed to affect approximately four to six million people worldwide. About 80% of PD patients develop dementia leading to PDD. Typically, dementia occurs late in the course of Parkinson's disease. In DLB, dementia is the first symptom, while motor symptoms may appear in the first year, making this the clinical distinction between these disorders. DLB may represent up to 15-20% of all dementia.

Alpha-synuclein is a small 140 amino acid intraneuronal protein, predominantly located presynaptically. Intraneuronal accumulation of a-synuclein results in the formation aggregates inside neurons, such as Lewy bodies and pale bodies, which are large round cytoplasmic inclusions, Lewy neurites, which are thread like inclusions in axons, and small synaptic inclusions.

The process leading to α-synuclein aggregation and toxicity is not well understood. Mutations or duplications of the a-synuclein gene are rare cause of PD and DLB. The pathogenic mutations A30P, A53T, E46K (Kruger et al., 1998) (Polymeropoulos et al., 1998) (Zarranz et al., 2004) and duplication and triplication of the α-synuclein (Chartier-Harlin et al. 2004) (Singleton et al., 2003) have been reported to cause either PD or DLB. Most of these mutations have been linked to increase in the rate of protofibrillar and finally fibrillar species are formed, and these may have different toxic properties. Aggregation is associated with phosphorylation of alpha-synuclein at serine 129. While normal alpha-synuclein is only phosphorylated to small extent (4%) the fibrillary alpha-synuclein is estimated to be 80% phosphorylated, and antibodies to pS129 alpha-synuclein detect the smallest aggregates.

Small amounts of alpha-synuclein are found extracellularly in interstitial fluid in brain, in Cerebrospinal fluid (CSF) and blood. The source of this alpha-synuclein is not clear, but it is likely released from cells, both from cell bodies and synapses, for example during synaptic release when neuronal cells are activated. Some of it is also likely coming from diseased and dead cells. Alpha-synuclein is also found in many blood cells and platelets, which are a likely source of alpha-synuclein in blood. Small proportion of alpha-synuclein is secreted in exosomes, while majority of alpha-synuclein in extracellular fluid is found as free protein not associated with exosomes.

There is a need for improved diagnostic tools to identify patients at early stages of a neurodegenerative disease with α-synuclein pathology. There is evidence that the neurodegenerative process starts years, perhaps 10-20 years before clinical diagnosis. REM sleep behaviour disorder (RBD) is now recognized as the prodromal stage of an α-synucleinopathy. Most people with RDB will convert to PD or DLB within the next 20 years from diagnosis of RBD. Other signs of prodromal stage of alpha-synucleinopathy include loss of olfaction.

Today there are no biochemical methods to aid a clinician's diagnosis before the motor symptoms are evident. At that point, substantial damage to the brain has probably already occurred. The importance of accurate diagnostic assays will become even greater as new disease modifying therapies emerge, that will hopefully stop or slow the progression of the disease.

Several assays measuring the total alpha-synuclein are available, and some of them are validated or are being validated in large clinical studies (Goldman et al. 2017). These studies have shown a small decrease in total alpha-synuclein in CSF to be associated with Parkinson's disease. Several exploratory assays to measure some kind of oligomeric forms of alpha-synuclein, and phosphorylated forms on Serine-129 also exist (Majbour et al. 2016a), and preliminary data suggests that these species may be increased in PD CSF. However, again the difference between patient and control levels are small and there is a large interindividual variation. All of the current assays suffer from the small difference and large interindividual variation, making these assays not useful in the help for diagnosis for diseases with alpha-synuclein aggregation. All in all, the current evidence suggests that total CSF α-synuclein is reduced in PD and that subspecies of α-synuclein oligomers, or phosphorylated alpha-synuclein may distinguish PD from controls.

Measuring alpha-synuclein in peripheral biofluids, blood plasma and saliva has produced even more variable results than CSF. In latest large study plasma and saliva α-synuclein levels did not significantly differentiate PD from healthy control participants and there was no significant correlation of a-synuclein levels in peripheral biofluids (plasma and saliva) with α-synuclein levels in CSF (Goldman et al. 2017). Therefore, currently CSF α-synuclein is of greater diagnostic utility for PD than peripheral α-synuclein. CSF sampling is more invasive way for obtaining samples, and it would be beneficial to have plasma biomarker for PD, if this was possible.

### SUMMARY OF THE INVENTION

Monitoring alpha-synuclein aggregation is of great importance for studying the pathogenesis of synucleinopathies, a group of neurodegenerative diseases that includes Parkinson's disease (PD), dementia with Lewy bodies (DLB), diffuse Lewy body disease (DLBD), and multiple system atrophy (MSA). Accordingly, the method of the present invention can be used to diagnose or monitor the disease progression of the diseases mentioned above. Alternatively, the method can be used to monitor or follow the treatment response and to take discussions related to treatment of the patients. This treatment may in certain embodiments be active or passive immunotherapy directed against alpha-synuclein, such as antibody treatment or vaccinations comprising alpha-synuclein.

In one embodiment, the present invention is an in vitro method comprising the steps of
a) Obtaining a sample from a patient
b) Applying the sample in a luminescence assay of the invention, and
c) Optionally, comparing the results with a control

The sample is a preferably a blood sample, a plasma sample or a serum sample.

The antibodies used are alpha-synuclein antibodies linked to a fluorophore. Two different fluorophore may be used in the method of the invention which may be linked to two antibodies binding to alpha-synuclein. One fluorophore has longer fluorescence time (donor) than the other fluorophore used (acceptor).

The donor is preferably selected from Lumi4-Tb (Tb2+ cryptate) or Europium cryptate (Eu3+ cryptate), and the acceptor is preferably selected from XL665, or fluorescein or d2. The proximity between the donor and acceptor is assessed by detecting the level of energy transfer by measuring the fluorescence emission, preferably at two different wavelengths such as 665 nm and 620 nm) in a compatible reader

### DRAWINGS

FIG. 1 shows measurements done using PHERAstar FSX device, which allows for simultaneous measurement of both 620 nM (Tb-cryptate—donor) and 665 nm (d2-acceptor) emissions. Ratio of 665/620*10000 is calculated for each well. The relative energy transfer rate, Delta F %, is calculated using the ratio and can be normalized to protein concentration. The column graph shows the Delta F % normalized to protein for each of the plasma sample (diluted eight times) measured using both kit #1 and kit #2 (detecting aggregated alpha synuclein). There is a clear distinction between the three PD patients and the three healthy controls. This figure shows that there is a significantly high level of aggregated alpha synuclein in the plasma of PD patients and this method can be used to identify and diagnose and monitor disease progression in PD patients based on a blood sample.

### DETAILED DESCRIPTION OF THE INVENTION

The object of the present invention is to provide an assay for use in diagnostic methods for alpha-synuclein related disorders, i.e., alpha-synucleinopathies wherein accumulation of aggregated insoluble alpha-synuclein in the form of Lewy bodies, Lewy neurites and small synaptic inclusions, are present in the brain. Such disorders include, but are not limited to, one or more neurodegenerative disorders such as Parkinson's disease (PD), Parkinson's disease with dementia (PDD), dementia with Lewy bodies (DLB), multiple system atrophy (MSA), and REM sleep behavioural disorder (RBD) and other neurodegenerative disorders with alpha-synuclein pathology.

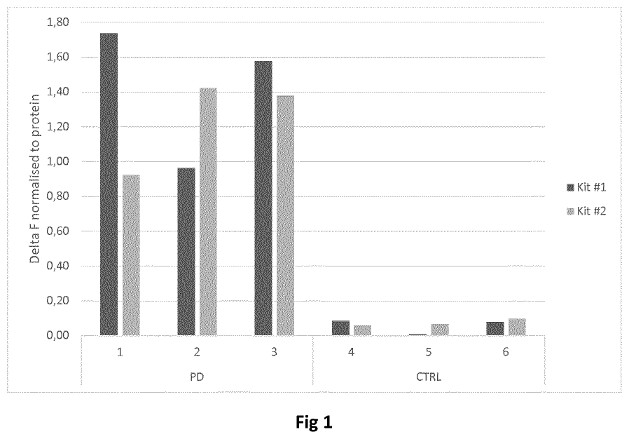

The present invention provides new use for two assays, one based on aggregated alpha-synuclein and one on phosphorylated alpha-synuclein. Both assays show large increase in plasma from PD patients compared to plasma from control individuals (FIG. 1).

According to one aspect of the invention, the invention relates to a method of treating a patient diagnosed as having alpha-synucleinopathies, such as Parkinson Disease, using the luminescence assay of the invention by treating said patients with an effective amount of an alpha-synuclein antibody.

The treatment effect of the antibody treatment may be evaluated measuring the patients level of alpha-synuclein in blood, plasma or serum. The evaluation may be made prior to antibody treatment (e.g. when diagnosing the patient) or after 1, 2, 3, 4 or more treatments. For example, the method may according to one embodiment also be used to monitor the treatment effect of the antibody treatment and the disease progression.

The assay may comprise the steps of

a. Applying a blood, plasma or serum sample from a patient to a luminescence assay under appropriate binding conditions, and

b. if applicable, comparing the data with a control from a person that do not have Parkinson's Disease or for example, comparing the data with data obtained at a different (earlier) timepoint from the same person, and thereby monitor the disease progression

Based on the result it may be determined whether or not the patient shall continue treatment, or if just diagnosed if the patient is to initiate treatment. For example, if the data obtained by the assay shows more alpha-synuclein present compared to the control continued or initiation of treatment may be advised.

The difference between the control and the patient may be more the 2-fold, 3-fold, 4-fold or above. As shown in FIG. 1, the control is very low and the thus a positive result may be found by disregarding the background.

The assay is an assay that measures luminescence may be a HTRF (Homogeneous Time Resolved Fluorescence) based assay. This technology combines fluorescence resonance energy transfer technology (FRET) with time-resolved measurement (TR) (Degorce et al, 2009, current Chemical Genomics, 3, 22-32). In the TR-FRET assays, a signal is generated through fluorescent resonance energy transfer between donor and an acceptor molecule (e.g. coupled to an antibody) when in close proximity to each other.

HTRF technology may use Europium cryptate as a fluorescence donor to monitor reactions between biomolecules such as antibodies or Terbium cryptate (Tb). Examples include Europium cryptate (Eu3+ cryptate) and Lumi4-Tb (Tb2+ cryptate).

An acceptor developed for HTRF may be XL665, a phycobiliprotein pigment purified from red algae. XL665 is a large heterohexameric edifice of 105 kDa, cross-linked after isolation for better stability and preservation of its photo physical properties in HTRF assays. A type of acceptor that possesses a series of photo physical properties very similar to those of XL665 but is characterized by organic structures which are 100 times smaller than XL665 are e.g. earth chelate or cryptate. By using smaller entities this solves the steric hindrance problems sometimes suspected in XL665 based TR-FRET systems. These near-infrared acceptors are also particularly suited for homogeneous assays since their emission is less likely to be disturbed by intrinsic medium or compound auto fluorescence arise in the typical compound screen process. The properties of these red acceptors also make them suitable for coupling with Terbium cryptate. Moreover, due to additional peaks in its emission spectrum, Terbium cryptate can be coupled with green acceptors such as fluorescein, emitting in the 520 nm range that may for instance allow designing multiplex assays with two readouts.

In particular, a fluorescent compound such as a rare earth chelate or cryptate will advantageously be used, especially a terbium, europium, dysprosium, samarium or neodymium chelate or cryptate. A terbium or europium cryptate will preferably be used.

In the fluorescent methods of detection and/or determination using the method of measurement of the invention, a rare earth cryptate described in European patent applications EP180 492 and EP321 353 will advantageously be chosen.

The terbium cryptate Tb trisbipyridine or the europium cryptate Eu trisbipyridine, as described in European patent application 180 492, or the cryptares Eu trisbipyridinediamine and Tb trisbipyridinediamine, described in European patent application EP321 353, will preferably be used.

According to an advantageous feature, the fluorescent donor compound is a europium cryptate and the fluorescent acceptor compound is selected from d2, allophycocyanin, allophycocyanin B, phycocyanin C or phycocyanin R.

It is also possible to use a phosphorescent compound, such as eosin or erythrosine, as the luminescent donor. In this case, it will be advantageous to use a fluorescent acceptor compound selected from chlorophylis such as those mentioned in European patent applications EP71 991 and EP314 406, or porphyrins such as those mentioned in European patent application EP71 991, or else phthalocyanins such as those of international patent application WO 88 04777.

According to another feature of the invention, the luminescent method for detecting and/or determining alpha-synuclein in a medium (e.g. blood, plasma or serum) comprise the steps of:

1) adding, to said medium containing alpha-synuclein from the patient or subject, a first antibody against alpha-synuclein, coupled with a luminescent donor,

2) adding a second alpha-synuclein antibody coupled with a luminescent acceptor,

3) incubating said medium after the addition of reagents

4) exciting the resulting medium at the excitation wavelength of the luminescent donor, and

5) measuring the signal of the luminescent donor at a wavelength (this measurement serving as a reference), and the signal resulting from the energy transfer at a different wavelength.

According to one embodiment, an assay is intended for the detection of alpha-synuclein aggregation using the HTRF technology. Aggregated alpha-synuclein is detected using specific alpha-synuclein monoclonal antibodies, labelled either a donor such as Tb-Cryptate or with an acceptor. When the dyes are in close proximity, the excitation of the donor with a light source (laser or flash lamp) triggers a Fluorescence Resonance Energy Transfer (FRET) towards the acceptor, which in turn fluoresces at a specific wavelength (665 nm). The antibody labelled with acceptor or Tb binds to alpha-synuclein. When alpha-synuclein aggregates, the antibody labelled with acceptor or Tb come then into a close proximity generating FRET. Signal intensity is proportional to the number of aggregates formed.

Monitoring alpha-synuclein aggregation is of great interest for studying the pathogenesis of synucleinopathies, a group of neurodegenerative diseases that includes Parkinson's disease (PD), dementia with Lewy bodies (DLB), diffuse Lewy body disease (DLBD), and multiple system atrophy (MSA). Accordingly, the method of the present invention can be used to diagnose or monitor the diseases mentioned above. Alternatively, the method can be used to monitor or follow the treatment response and to take decision related to treatment of the patients. In one embodiment, this treatment is alpha-synuclein antibody treatment.

Thus, the present invention relates to method of diagnose or follow synucleinopathies such as Parkinson Disease in patients comprising the steps of
a) Obtaining a sample from a patient
b) Applying the sample on a luminescence assay, as described above, and
c) Optionally, comparing the results with a control

The sample is a preferably a blood sample, a serum sample or a plasma sample

The antibodies used may be alpha-synuclein antibodies linked a fluorophore. The fluorophore used has a longer fluorescence time (donor) than the other fluorophore used (acceptor). The donor is preferably selected from Lumi4-Tb (Tb2+ cryptate) or Europium cryptate (Eu3+ cryptate), and the acceptor is preferably selected from d2, XL665, or fluorescein.

The proximity between the donor and acceptor is assessed by detecting the level of energy transfer by measuring the fluorescence emission, preferably at two different wavelengths (such as 665 nm and 620 nm) in a compatible reader

As used herein, the term “alpha-synuclein” is synonymous with “the alpha-synuclein protein” and refers to any of the alpha-synuclein protein isoforms (identified in, for example, UniProt as P37840, 1-3). The amino acid numbering of alpha-synuclein is given with respect to the sequence as shown below, with methionine (M) being amino acid residuel (SEQ ID NO: 1):

MDVFMKGLSK AKEGVVAAAE KTKQGVAEAA GKTKEGVLYV
GSKTKEGVVH GVATVAEKTK EQVTNVGGAV VTGVTAVAQK
TVEGAGSIAA ATGFVKKDQL GKNEEGAPQE GILEDMPVDP
DNEAYEMPSE EGYQDYEPEA

The alpha synuclein antibodies may in some embodiments be binding the C-terminal residues between 120-140 of alpha synuclein or pS129 alpha-synuclein.

The present invention also relates to a method of treating a Parkinson Disease patient by administering an effective amount of an antibody binding an epitope on alpha-synuclein, wherein the patient has been diagnosed or is monitored by the assay of invention.

### EXAMPLE

This assay can be used to identify, diagnose and monitor patients suffering from alpha synucleinopathy, e.g. Parkinson Disease, using a blood sample.

Assay principle:

This assay is based on using a Time resolved-Fluorescence Resonance Energy Transfer, (TR-FRET) technology.

FRET is based on transfer of energy between two dyes, a donor and an acceptor. When the donor is excited, it transfers energy to the acceptor which in turn emits fluorescence that is measured. This is only possible if the dyes are very close to each other. Normal FRET studies are limited by background fluorescence which are extremely transient. This can be overcome by combining Time resolved measurements with FRET to avoid background and non-specific fluorescence. Also, the acceptors in TR-FRET are designed to emit long-lived fluorescence when they are involved in FRET.

In the alpha synuclein aggregation kit, a specific monoclonal antibody is labelled with either a donor or an acceptor molecule. In the Phospho-synuclein kit (S129P), two antibodies are used, one is an alpha synuclein antibody and the other a Phospho-synuclein specific antibody (S129P), where one of the antibodies is labelled with a donor and the other with an acceptor dye. When the antibodies (labelled with donor and acceptor dyes) bind to human alpha synuclein aggregates, they come very close to each other and generate FRET upon excitation.

Patients

Patients were recruited from the outpatient clinic of the Department of Neurology, Bispebjerg-Frederiksberg Hospitals. The clinical diagnosis for PD was defined according to UK Parkinson's Disease Society Brain Bank clinical diagnostic criteria. Patients were included consecutively and a clinical follow-up was performed for all patients. At follow-up, only those patients fulfilling the diagnostic criteria for a definite PD diagnosis were accepted in the study. Subjects from the control group were free of diseases that might affect the central nervous system.

Plasma Samples

Venous blood was drawn at the respective clinics and processed on the same day at Bispebjerg Movement Disorders Biobank, Copenhagen, DK. All plasma samples were collected at inclusion. Samples were collected in EDTA coated polypropylene tubes, and were spun at 2000×g for 10 minutes at 4° C.; the supernatant plasma was then aliquoted and stored in 400 μL polypropylene (PP) tubes at −80° C. until the day of analysis, when they were thawed on ice for 30 min.

### Example 1

Samples from patients (3× controls and 3× PD pts) were analyzed. Two commercial kits from Cisbio were used, alpha synuclein aggregation kit (Kit #1) and a Phospho-synuclein kit (Kit #2), to compare the samples.). Each sample was tested in three different dilutions. The samples were diluted in the buffer provided with the kits and pipetted into a 386 well plate in duplicates. The antibody mixture is added to the well and incubated at RT. Positive and negative controls are included in the plate.

Measurements were done using PHERAstar FSX device, which allows for simultaneous measurement of both 620 nM (Tb-cryptate—donor) and 665 nm (d2-acceptor) emissions. Ratio of 665/620*10000 is calculated for each well. The relative energy transfer rate, Delta F %, is calculated using the ratio and can be normalized to protein concentration. The column graph shows the Delta F % normalized to protein for each of the sample (diluted eight times) measured using both kit #1 and kit #2. There is a clear distinction between the three PD patients and the three healthy controls. This example shows that there is a significantly high level of aggregated alpha synuclein and phospho-synuclein in the plasma of PD patients and this method can be used to identify and diagnose PD cases based on a blood sample.

1. A method of detecting alpha-synuclein in a sample of a subject comprising the steps of:
a) Obtaining a sample from a subject,
b) Applying the sample to a luminescence assay, and
c) Optionally, comparing the results with a control.

2. The method according to claim 1 wherein in said sample is a blood sample, a plasma sample or a serum sample.

3. The method according to claim 1, wherein the luminescence assay is using alpha-synuclein antibody binding within the 120-140 residues on alpha-synuclein or pS129 alpha-synuclein.

4. The method according to claim 1, wherein the luminescence assay is using two different alpha-synuclein antibodies binding different epitopes within the 120-140 residues on alpha-synuclein or pS129 alpha-synuclein.

5. The method according to claim 3, wherein one fluorophore used has a longer fluorescence time (donor) than the other fluorophore used (acceptor).

6. The method according to claim 5, wherein the donor fluorophore is selected from Lumi4-Tb (Tb2+ cryptate), Europium cryptate (Eu3+ cryptate).

7. The method according to claim 5, wherein the acceptor fluorophore is selected from d2, XL665, or fluorescein.

8. The method according to claim 1, wherein the proximity between the donor and acceptor is assessed by detecting the level of energy transfer by measuring the fluorescence.

9. The method according to claim 1, further comprising wherein the subject is administered an effective amount of an antibody against alpha-synuclein if the method shows alpha-synuclein levels in the sample that are higher than a control sample from a healthy subject.

10. The method according to claim 1, wherein the method is used to diagnose or monitor alpha-synucleinopathies.

11. The method according to claim 1, wherein the method is used to monitor the treatment response of a medicament used to treat alpha-synucleinopathies.

12. The method according to claim 1, wherein the subject is a human.

13. A method of treating a Parkinson Disease patient comprising administering to the patient an effective amount of an antibody binding an epitope on alpha-synuclein, wherein the patient has been diagnosed or is monitored by the assay of claim 1.


SEQUENCE LISTING (1 sequence)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA